(19) United States
(12) Patent Application Publication
(10) Pub. No.: US 2025/0040865 A1
(43) Pub. Date: Feb. 6, 2025

(54) Autonomic Nervous System Diagnostic and Therapeutic Device for Corrective Therapy

(71) Applicants: David Gerdt, Faber, VA (US); Tyvin Rich, Charlottesville, VA (US)

(72) Inventors: David Gerdt, Faber, VA (US); Tyvin Rich, Charlottesville, VA (US)

(21) Appl. No.: 18/679,164

(22) Filed: May 30, 2024

Related U.S. Application Data
(60) Provisional application No. 63/469,700, filed on May 30, 2023.

(51) Int. Cl. A61B 5/00; A61B 5/0205; A61B 5/024; A61B 5/08; A61B 5/16; G16H 20/70

(52) U.S. Cl. CPC A61B 5/4035; A61B 5/02405; A61B 5/08; A61B 5/165; A61B 5/4836; A61B 5/6814; A61B 5/6824; A61B 5/6826; A61B 5/6829; A61B 5/7257; A61B 5/742; G16H 20/70; A61B 5/0205; A61B 2560/045; A61B 2560/0462; A61B 2562/0247

(57) ABSTRACT

The disclosed system relates to the diagnosis and correction of autonomic nervous system imbalance through the recognition and improvement of parasympathetic tone of a user using paced breathing and vibratory neuromodulation. The system includes a wearable IBI detector, software for analyzing PSD data and determining recommended therapy, a vibratory stimulator, and optional smart phone guidance. The IBI detector reads the pulsatile waveform to produce IBI data that is used to compute PSD data. The PSD data is analyzed for ANS imbalance indicators and therapy is recommended based on continuously updated information stored in a database. Results from application of recommended therapy are stored and shared and can be used to build a personalized training regime to strengthen parasympathetic tone.

### FIELD OF THE INVENTION

The invention relates to a device for recognizing and improving parasympathetic response of a user through paced breathing and neuromodulation.

### BACKGROUND OF INVENTION

Vasomotor symptoms (VMSs) affect more than 2.5 million menopausal women and cancer patients per year in the U.S. causing sleep loss and reduced productivity. The disclosed wearable detector provides precise detection of inter-beat intervals (IBIs) and uses them for the personalized management of VMSs. Data (95%) shows measuring pulse arrival times results in distinguishing power spectral densities (PSDs) to measure and interpret sympathetic and parasympathetic cardio-nervous system power, activity, or tone which can improve or eliminate many symptoms relating to a number of diseases

The autonomic nervous system (ANS) controls involuntary, unconscious processes such as heart rate, blood pressure, respiration, and digestion and voluntary processes such as breathing rate and is composed of two main parts: the sympathetic nervous system and the parasympathetic nervous system. The sympathetic nervous system is often thought of in terms of the flight-or-fight response, i.e., body processes that help prepare the body for strenuous physical activity in times of need, especially stress and danger. The parasympathetic nervous system is often thought of in terms of rest-and-digest response, i.e., body processes that relax your body after periods of stress or danger and aid in life-sustaining processes.

The complexity and stress of ageing in modern western lifestyle can frequently overwhelm our coping mechanisms resulting in the increased prevalence of anxiety, depression, and hypertension as well as many chronic illnesses and medical conditions seen today. These ailments indicate, in part, that an overactive sympathetic nervous system and/or suppressed parasympathetic nervous system contribute to mental and physical health issues. Likewise, an overactive parasympathetic tone may suggest other unbalancing of the ANS.

### SUMMARY OF THE INVENTION

A device and system for recognizing and correcting autonomic nervous system imbalance and strengthening improving parasympathetic tone in vertebrates comprises a wearable detector with a body substantially comprised of at least one flexible substrate. In addition to the substrate the device contains a bladder, a pressure sensor, a detector microprocessor with communication means, a power source member, an exterior side and a user contact side. The substrate, or other securing member, is configured securely maintain the device adjacent to at least one of a thumb, a finger, a wrist, an ankle, or scalp.

In use the bladder is placed adjacent an artery and secured in place by a securing member to measure pulsatile waveform and detect inter-beat intervals (IBI) from the artery, for the IBI data to be read pressure sensor. The IBI data received from the pressure sensor and sent to the detector microprocessor is used to compute power spectral density (PSD) data. This PSD data reflects an evolving autonomic nervous system spectra for evaluating parasympathetic tone.

The device communicates with at least one external device including a user device, a stimulation unit and respiration monitor, all of which may be communicative coupled with one another. The stimulation unit has a mechanical vibrational element placed at a vagus nerve and a heart rate variability monitor with a stimulation unit microprocessor and communication means to receive data from the detector microprocessor or the user device. The stimulation unit microprocessor controls application of vibratory stimulation based on received data including when to engage stimulation, duration of stimulation, intensity of stimulation and frequency of stimulation.

Communication between the detector microprocessor and any external devices can be wired, Bluetooth, WiFi, NFC, RFID, or other wireless communication.

In one embodiment the at least one flexible substrate is translucent, and the device further comprises a thumb pump, LED and closure members. In this embodiment the bladder, pressure sensor, LED and detector microprocessor are placed on, and welded to, the user contact side. The pressure sensor is in communication with the bladder to read the pulsatile waveform within the bladder and transmit the IBI data to the microprocessor. The LED is also connected to the pressure sensor and indicates to the user when the bladder reaches usable pressure to detect the inter-beat intervals. The detector microprocessor receives IBI data from the pressure sensor, analyzes the IBI data and communicates this data to a predetermined remote microprocessor. A power source, generally a small battery is used to power the detector microprocessor and LED. On the exterior side the thumb pump is positioned over the bladder and used to expand the bladder to the usable pressure. Once the bladder is placed adjacent the artery the closure members interact with one another to affix the device in position.

In another embodiment the flexible substrate is translucent with an LED and external securing means. The user contact side of the substrate has the bladder, pressure sensor, LED, microprocessor and power source. The pressure sensor is printed onto the bladder to read the pressure within the bladder and when the bladder reaches usable pressure to detect the inter-beat intervals, the LED is activated. The detector microprocessor receives the IBI data from the pressure sensor, analyzes the data and communicates the IBI data to a predetermined remote microprocessor. A battery serves as the power source.

Once the bladder is placed on the artery initial tightening is facilitated through use of a tab at one end of the substrate and a slot at the opposing end to receive the tab. External securing means, such as clamps or zip ties, then are secured around the wearable detector.

In another embodiment the device is composed of multiple flexible, translucent, substrates. A first of the substrates contains the bladder welded onto a first surface; the a second of the substrates contains the charging members on a first surface; and a third of the substrates contains the pressure sensor, the microprocessor, LED, power source and a second communication member on a first surface. The third substrate can extend beyond the first and second substrates to create a securing member. To assemble the first side of the third substrate is placed adjacent the second side of the second substrate and the first side of the second substrate is placed adjacent the second side of the substrate to form a detector which is then sealed for waterproofing. The LED is connected to the pressure sensor to indicate when the bladder reaches usable pressure to detect the inter-beat intervals, and the data from the pressure sensor is sent to the microprocessor and then to a remote microprocessor.

In an additional embodiment the flexible, translucent substrates are layered to form the device. The first of the substrates has a first periphery and contains the bladder welded onto the first surface and an air channel extending through to the second substrate. The second substrate has a second periphery which is less than the first periphery and contains the pressure sensor positioned adjacent the air channel, a microprocessor, LED, power source and on/off switch. The third substrate has a U-shaped configuration with flat rim having a first periphery and an interior periphery dimensioned to receive the second substrate. The flat rim of the third substrate is placed adjacent a second side of the first substrate and then sealed for waterproofing. As with prior embodiments the LED is connected to the pressure sensor to indicate when the bladder reaches usable pressure to detect the inter-beat intervals, and IBI data from the pressure sensor is sent to the microprocessor for analysis and then to a remote microprocessor.

In still another embodiment two layers of the flexible, translucent substrates are used for the device. A first substrate has a first periphery and contains the bladder welded onto a first surface and an air channel extending there through. A second substrate contains the pressure sensor positioned adjacent the air channel, the microprocessor, LED, power source and on/off switch. The first side of the second is placed adjacent a second side of the first substrate and then sealed for waterproofing.

The LED is connected to the pressure sensor to indicate when the bladder reaches usable pressure to detect the inter-beat intervals, and IBI data from the pressure sensor is sent to the microprocessor for analysis and then to a remote microprocessor.

As a system, consisting of a wearable detector, vibratory stimulator and analysis software, preferably run by a computer application on an electronic mobile user device, a wearable detector is configured to continuously detect IBIs from a user and compute PSD data from the IBIs using at least one pulse pressure sensor and at least one microprocessor. The IBIs are obtained through an observation period in the range of about 3 to 5 minutes at a sampling frequency of at least 300 Hz, with the PSD data comprising an evolving autonomic nervous system (ANS) spectra. A vibratory stimulator configured to deliver vibrations to vagus nerve endings of the user's ear contains a microprocessor in communication with the wearable detector. A user device, such as a smart phone, is communicatively coupled to at least the wearable device and configured to run applications, display information on a graphical user interface, store information in at least one database locally and remotely, and transmit information.

One or more processors communicatively coupled with at least the wearable detector and the user device with the one or more processors is configured to transmit PSD data to display on the user device providing updated ANS status information to the user;
analyze PSD data for indicators of ANS imbalance correlating to one or more health-related conditions, the indicators comprising one or more of the following: change in low frequency power (LF) to high frequency power (HF) ratio, frequency power readings within a pre-determined pattern, and erratic frequency power readings. The one or more health-related conditions can include, among others, vasomotor symptoms, anxiety, and PTSD;
compare indicators to information stored in said at least one database;
determine recommended therapeutic action based on information stored in the at least one database, with the recommended therapeutic action comprising one or more of the following: paced breathing, for example paced breathing at 10 bpm, with or without vibrational stimulation of the vagus nerve, vibration of the vagus nerve during exhalation to strengthen HF, vibrational stimulation of the vagus nerve, and neuromodulator application;
transmit instructions for recommended action to at least display on the user device;
receive additional PSD data for continued analysis during application of therapeutic action; and
record ANS changes based on said additional PSD data and save ANS changes to said at least one database.

Optionally one of the processors can be communicatively coupled to the vibratory stimulator and configured to transmit instructions for recommended action to said vibratory stimulator to automatically prompt vibrational stimulation of the vagus nerve.

In some embodiments a respiratory monitor transmits respiration data to the processors with the respiration data used to determine type and timing of recommended therapeutic action.

### BRIEF DESCRIPTION OF THE DRAWINGS

FIG. 1 is a representation of an example placement on a body of a stimulation system in accordance with the invention;

FIG. 2 is a drawing of an example of an over the ear stimulator in accordance with the invention;

FIG. 3 is a representation of the placement of the ear stimulator, in accordance with the invention;

FIG. 3a is a representation of the location of the Marvelous Point, in accordance with the invention;

FIG. 4 is a representation of the placement of the ear stimulator and ear clip in accordance with the invention;

FIG. 5 is a plan view of the external side of an embodiment of an example finger cuff, in accordance with the invention;

FIG. 6 is a side view of the finger cuff of FIG. 5 in accordance with the disclosed invention;

FIG. 7 is a plan view of the user contact surface of an alternate embodiment of a finger cuff in accordance with the invention;

FIG. 8 is a side view of the finger cuff of FIG. 7 in accordance with the disclosed invention;

FIG. 9 is a plan view of the finger cuff of FIG. 7 positioned as it would be on a thumb and including the retaining ring 320 and tightening element 322, accordance with the disclosed invention;

FIG. 10 is an exploded of a waterproof embodiment of the finger cuff, accordance with the disclosed invention;

FIG. 11 is a side view of the assembled finger cut of FIG. 10, in accordance with the disclosed invention;

FIG. 12 is cutaway side view of another embodiment of an assembled finger cut, in accordance with the disclosed invention;

FIG. 13 is a plan view of the circuit board of FIG. 12, in accordance with the invention;

FIG. 14 is an exploded view an additional embodiment of the finger cuff, in accordance with the disclosed invention;

FIG. 15 is an overview flow chart of data gathering in accordance with the invention;

FIG. 16 is an overview flow chart of the therapy system of the present invention.

FIG. 17 is a flow chart of method of analysis and recommendation of the present invention.

FIG. 18 is graph illustrating PSD at a normal breathing rate.

FIG. 19a is a graph illustrating PSD at 10 bpm for 5 minutes.

FIG. 19b is graph illustrating PSD at 10 bpm for 5 minutes plus VsB.

FIG. 20 is a graph showing HF drop during hot flash.

FIG. 21 is a graph showing PSD at 6 bpm.

FIG. 22 is a graph showing paced breathing observations of blood pressure and heart rate.

FIG. 23 illustrates an example graphical user interface of the computer application that supports ANS therapy.

FIG. 24 is a flow chart of the steps for the user can self-guide through a chosen technique.

### DETAILED DESCRIPTION OF THE INVENTION

### Definitions

As used herein the term “power” shall refer to an integrated set of amplitudes in the power spectrum analysis (PSA). The variance is measured between successive heart beats and, after mathematical transformation, results in the power spectral density.

As used herein the term “variance” shall refer to a mathematical calculation based on the arrival time difference between successive heart beats, the mean of all the variations, and the number of differences. The square of the variance equals the sum of all the variances minus the mean of the variances, everything divided by N, the number of variances. The square root of the variance is the standard deviation.

As used herein the term “power spectrum analysis” (PSA) or “spectral analysis” shall refer to a technique applying a Fast Fourier Transform (FFT), Auto Regression Analysis (AR) or other equivalent mathematical transformations to the variation of a particular signal to compute its power versus frequency spectrum. The result is presented as a plot of statistical power, rather than signal power, against frequency and is referred to as its power spectrum.

As used herein the term “Fast Fourier Transform” (FFT) shall refer to an algorithm that converts a signal into a linear sum of individual frequency components with their powers or amplitudes and thereby provides frequency information about the signal such as overactivity of the sympathetic nervous system (NS).

As used herein the term “Power Spectral Density” (PSD), energy, and power are interchangeable and shall refer to an indication of how much variance (or power) there is in each set of frequencies within a five-minute or other timed observation window. The set of frequencies that would, when added, produce the level of power, at each frequency in the variance as well as frequencies where the blood pressure and heart rate are oscillating.

As used herein the term “frequency” shall refer to the number of cycles per second in the power spectral density (PSD).

As used herein the term “HR detector” shall refer to any wired or wireless vertebrate monitoring system such as Fitbit, iWatch, VitalStream by Caretaker Medical, or other device having the ability to accurately detect the time of arrival of heart rate on a beat-by-beat basis.

As used herein the term “gating” shall refer to the mechanism by which spinal gates open or close, thereby allowing or limiting the transmission of pain. [APA Dictionary of Psychology]

As used herein “coherence” shall refer to a condition where the blood pressure and heart rate oscillate at the same frequency as the respiration rate or the neuromodulator rate.

As used herein “IBI” (inter-beat intervals) shall refer to the time between adjacent heartbeat pulses traveling along an artery and is the reciprocal of the heart rate.

As used herein “HRV” (heart rate variability) shall refer to the set of variations (variance) between successive heartbeats.

As used herein the term “IBI detector” shall refer to any device that can detect the inter-beat interval with sufficient accuracy, including but not limited to a plethysmography devices such as VitalStream, an electrocardiogram or ECG, a photo-plethysmographic (PPG), such as made by HeartMath, radar, or thermal, optical, acoustic, or electrical sensors.

As used herein “vagal tone” or “parasympathetic tone” refers to the activity of the vagus nerve, which is a fundamental component of the parasympathetic branch of the autonomic nervous system and frequently used to assess emotional regulation and heart rate functions. Vagal tone tells us how well the vagus nerve is functioning, and it is measured indirectly by heart rate variability (HRV).

As used herein the term “welding” shall refer to chemical welding, ultrasonic we could mean chemical welding, ultrasonic welding, electromagnetic welding, 3-D printing and sealing, or compatible adhesives.

As used herein the term “tone” shall refer to the activity or excitation level of nervous system, which in turn equals the power level in PSD that in turn provides a measure of an ability to respond to threat a condition like VMSs, drug addition, RA, dementia, inflammatory bowel disease and other dysautonomia.

Disclosed is a non-pharmacologic intervention that can correct autonomic nervous system (ANS) imbalances and specifically increase the parasympathetic tone through vibrational stimulation and paced breathing when there is a reduction of expected spectral distribution in the parasympathetic spectra. Such intervention is a useful treatment for a number of health issues, including but not limited to advanced warning of oncoming temporary (or permanent) dysautonomia, for example “hot flashes.” Hot flashes are vasomotor symptoms resulting from a specific dysfunction associated with hormone withdrawal. While often associated with menopause in women, other medical conditions such as thyroid disease, infection, addictive withdrawal, or cancer can also cause hot flashes in both men and women. Treatment by vibrational stimulation may be applied responsively or preventively. Responsive treatment is applied immediately upon detection of an unwanted change in spectral distribution by heart rate variability-power spectral density analysis software. Preventive treatment is applied via dosing at regular intervals which strengthens or trains the vagus nerve leading to sustained increases in the parasympathetic or sympathetic tone depending on the regimen. Stimulation dosing can be applied several times a day as needed for an individual's care.

A primary purpose of the disclosed system is to correct autonomic nervous system imbalance which involves a shift of the sympathetic/parasympathetic balance. Balance in this sense is an acceptable and often desirable distribution of frequencies, not necessarily an equality or balance in the sense of an analytical balance. The balance between the sympathetic nervous system and peripheral nervous system is reflected in an analysis of the high (HF) and low (LF) frequency power bands of the sympathetic and parasympathetic nervous system. Homeostasis is related to wellbeing and is advanced here to be a condition of shared spectral power between LF and HF, often called balance in a healthy state, but they are not necessarily equal in terms of spectral power within each band. Balance does not mean equality, but a distribution that works for the subject.

The vagus nerve is the set of main nerves in the parasympathetic nervous system which controls many involuntary functions such as heart rate, immune system, but is mainly a sensory nerve since it is composed of 80% afferent fibers carrying signals from the body to the brain. Vagal tone is the activity of the vagus nerve. Stimulation of the vagus nerve increases vagal tone and provides several benefits ranging from minimizing epileptic seizures to regulating emotions. Stimulation of the vagus nerve through an electrical set of pulses at the ear boosts the HF power in the HRV spectrum (the PSD). In U.S. Pat. No. 8,428,719 B2 to Napidow, the reduction of pain through electrical stimulation via pulses at the ear during exhalation was noted to increase HF-HRV. Additionally, applications of electrical stimulation of the vagus nerve have been found at Hampton University Proton Therapy Institute to lower LF/HF ratios in men. No one to date has used vibration to achieve this result although there are data that vagal stimulation with a vibrator will affect an arm of the vagal network referred to as the “inflammatory reflex” described by Addorisio, et al. (Addorisio M E, Imperato G H, de Vos A F, Forti S, Goldstein R S, Pavlov V A, van der Poll T, Yang H, Diamond B, Tracey K J, Chavan S S. Investigational treatment of rheumatoid arthritis with a vibrotactile device applied to the external ear. Bioelectron Med. 2019 Apr. 17; 5:4. doi: 10.1186/s42234-019-0020-4. PMID: 32232095; PMCID: PMC7098240).

HRV declines with age naturally. However, abrupt changes in HRV can be correlated with the onset of disease. The HRV that is currently reported commercially to the masses is the root mean square of successive differences between normal heartbeats. Changes in HRV herein refers to changes in the variability of beat-to-beat intervals, as the disclosed is not so concerned with time related parameters derived from these differences but have instead transformed the description into new parameters describing the interval differences in frequency space. When disclosed herein changes in HRV are referencing changes in the PSD. The differences can also be referred to as time domain HRV (HRV) and frequency domain HRV.

Many diseases have known loss of the parasympathetic tone, and this is the main focus of the disclosed system by having an approach by which too much or too little sympathetic output can be measured. The intervention can be prioritized by the disclosed system after determining the PSD in each of the LF or HF, and guided therapies are used to restore the PSD to what is normal for that person. In some embodiments, the disclosed system monitors the ANS by tracking IBI and making HRV and PSD calculations, recognizing a deficiency in the total integrated power of either the HF spectral band or the LF spectral band, and initiating stimulation. In other embodiments, the system recognizes the imbalance, transmits the data to a smart device that, in turn, provides guidance to the user for actions to take to stimulate the vagus nerve and balance the spectral bands. As an aid to the teaching of meditation, a PSD generator can confirm, without an instructor, that a certain meditative state has been reached.

In comparison to known electrical stimulation of the vagus nerve to treat conditions such as depression, epilepsy, and cardiac issues, the vibrational application is non-invasive, comfortable, easier to apply, and less expensive to implement in both clinical and home use. Using the disclosed system rather than an electrocardiogram eliminates the need for electrodes, thus eliminating the need for patients to partially disrobe, making it more friendly, easier to use, and simpler to operate. Additionally, there is no loss of chest hair or skin, which sometimes happens when an electrode is removed, particularly with the elderly. Further the disclosed finger sensor with pulse works independent of the pigmentation of the skin, a hindrance to PPG detectors.

The disclosed system focuses on improving the health and longevity of women, particularly those affected by menopause and experiencing vasomotor symptoms (hot flashes and sleep disruption). By reducing or eliminating VMSs, the system aims to help women return to work more promptly, resulting in improved productivity and longer work spans. Additionally, the system's impact on chronic conditions such as osteoporosis could further enhance the overall societal value by enabling women to contribute to society for longer periods and live more active lives.

### Apparatus

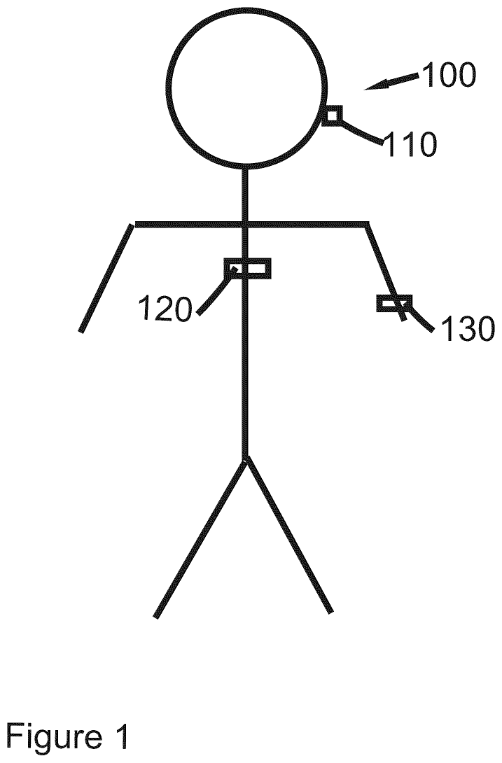

The system of the present invention is directed to a wearable IBI detector in communication with a vagus nerve vibratory stimulator, an external user device, and an optional respiration monitor. FIG. 1 illustrates the overall placement of an example of the disclosed system 100 using a wrist IBI detector 130 as the wearable detector, a chest band 120 as the respiration monitor, an ear stimulator unit 110 as the vagus nerve vibratory stimulator, and a cell phone 150 as the external user device.

The wearable detector must be placed at a location providing accurate pulse readings for accurate IBI detection. Placement on the thumb, finger, wrist, scalp, or ankle are viable options, with placement on the thumb or finger being most preferred. The detector must be enlarged for use on the wrist, upper arm, or ankle. Only the retaining portion of the detector will require resizing while the actual monitoring electronics can remain the same.

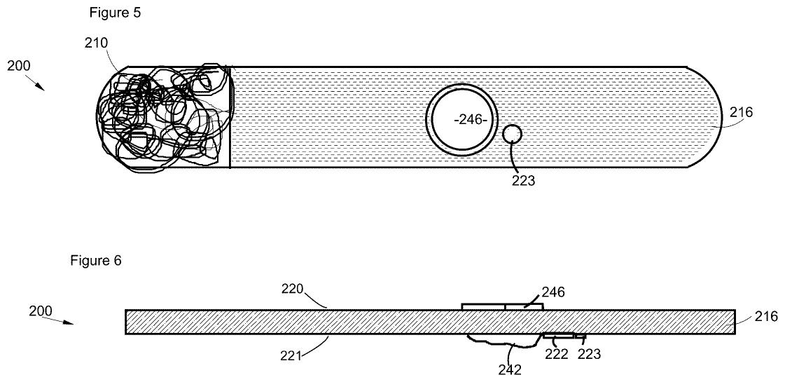

Regardless of the placement, the wearable detector contains a bladder, one or more pressure sensors, a microprocessor with communications means, and a power source. These features are shown in FIGS. 5-7 illustrating one example embodiment of a wearable IBI detector in the form of a finger cuff 200 having a body substantially comprised of a substrate 216 preferably placed surrounding the proximal phalanx of the thumb. Substrate 216 is configured similarly to an adhesive strip or one using hoop and loop attachment as long as all parts touching skin are skin-friendly polyurethane. The substrate 216 contains a flexible printed circuit board having a microprocessor with communication means (not shown) with a thin protective covering along the surface with the exception of the hook and loop material. The finger cuff 200 is retained in position using hook material 210 and loop material 212, attached to the substrate 216, making it easy to remove and reapply. Although in most applications the ability to reuse the finger cuffs disclosed herein is preferable, in some instances disposability would be advantageous and the substrate 216 can be replaced with an adhesive bandage material or printed polyurethane.

Finger cuff 200 has an interior 221 and an exterior 220. Interior 221 contains a bladder 242, a pressure sensor 222, and LED 223. Exterior 220 of the finger cuff 200 contains the thumb pump 246 that is used to inflate the bladder 242, increasing tactile communication between the artery and the bladder 242 by exteriorizing or bringing the pulse pressure waveform to the skin's surface. LED 223, as described hereinafter, serves to indicate that a usable pressure to the bladder 242 has been obtained.

In use the cuff 200 is wrapped tightly around the base of the thumb to enable the arteries to be unloaded thereby bringing the pressure pulse to the surface of the skin, adjacent the bladder 242. The pressure in the bladder 242 is pressurized through use of the thumb pump 246 to squeeze the air out of the finger/bladder interface and band interface to provide a proper pressure. The appropriate tightness can be determined, for example, using an LED 223, connected to the pressure sensor 222, that flashes a predetermined number of times when the pressure is correct. The determination of sufficient pressure can be by using a MEMS pressure sensor 222, or its equivalent, or by observing an acceptable pulse identification.

A pulse goes by every second or so, and there are two arteries in each finger or thumb. The blood pressure minimum is diastolic, and from the diastolic floor a pulse will rise to pressures as high as systole then begin declining in pressure as the pulse passes by. The passage of this pulse expands a portion of the artery outwardly like a sudden bulge, which in turn expands the tissue around the arteries, and which pushes into the bladder 242 raising the pressure within the thumb. The pressure sensor 222 measures the pulsatile waveform and changes in pressure from systolic to diastolic and back again, continuously accessing the pulse, at approximately 500 times a second. The pressure in the bladder is well below diastolic pressure and does not affect circulation or cause discomfort. The constant monitoring of the pulse achieves a high-quality digital representation of the analog data. The collected data is then used to infer various physiological parameters.

The hydraulic pressure variations of which the pulse is composed is followed by observing air pressure variations in the bladder due to the pulse passing by.

The foregoing elements are placed on a substrate 216, preferably breathable and non-allergenic, that can be manufactured in sizes appropriate for the end user. Optimally the cuff would be in the range of ⅙ inch thick, however this would vary depending upon the sizing of the elements. One method of manufacture of most parts would be urethane welding. Printed circuit boards and 3D printing can also be used for many of the parts along with other low-cost methods known in the art. A covering can be placed using printing methods over the exposed elements thereby protecting the elements and permitting extended use.

The finger cuff 200 illustrated in FIGS. 5-7 shows all elements, with the exception of the thumb pump 246, on the interior of the substrate 216 adjacent the user's thumb. Although this is preferable, this placement may not be possible in smaller sized cuffs for very small hands or children at which time a design moving pressure sensor 222 and LED 223 to the exterior of the cuff can be used. The LED 223 can remain on the interior contact side of the substrate 216 when a translucent polyethylene is used as the substrate material as the light will show through. In instances where the substrate 216 is not translucent, the LED 223 will need to be moved to the exterior. However, the bladder 242 must remain on the interior of the cuff in contact with the skin as the contact between the thumb pump 246 and the bladder 242 is pneumatic.

The finger cuff 200 provides an advantage over prior art monitoring devices in that it can be worn all day or night and can transmit gathered data, including the PSD spike achieved during coherence, to any connected smart device, such as a cell phone. In this embodiment, size prevents display on the device itself and data is wirelessly transmitted to a smart phone, smart watch, or fitness tracking device. The smart device would also be used to re-program the finger cuff 200. Updated algorithms can be passed from a cloud to the phone using over the air communications and the same OTA can be used to re-program the microprocessor in thumb device.

As previously noted, the cuff can be enlarged for use on the wrist, upper arm, or ankle. Only the retaining portion of cuff will require resizing while the actual monitoring electronics can remain the same. Depending upon the location, the size and end location, the materials of the substrate may need to be changed. The same monitoring elements would be incorporated into the larger cuffs enabling, in the larger sized embodiments, the addition of a screen to eliminate a secondary device for visual monitoring.

The technology, when preprogrammed with specific species parameters, can be used on most, if not all, mammals and avoids the problems of PPGs which involve excessive batteries, non-workability on darker skin, lightning hazards from the wires of necessary large batteries, and wires that introduce choke hazards such as the system designed by HeartMath.

The wearable IBI detector receives pulse pressure data through the pressure sensor(s) during an observation period of about 5 minutes at a sampling frequency of at least 300 HZ and transfers the data to the microprocessor to determine the IBI using a known algorithm and IBI detection techniques. The IBI data is stored on the microprocessor for about four minutes and is used by the microprocessor to compute PSDs using a customized FFT-based algorithm. Older IBI data is dropped out and replaced with new IBI data to continuously compute updated PSDs. While the microprocessor of the IBI detector may also be able to analyze the PSDs, this analysis is more commonly carried out by a computer application on the user's smart device.

PSD data is transmitted by the communication means of the wearable detector microprocessor to the user's external device for analysis. Communication between any elements of the present invention can be accomplished through wires or, preferably, wireless technology such as Bluetooth, WiFi, NFC, etc.

In addition to external user device communication, the wearable detector may also have the ability to communicate with the vibrational stimulator unit and respiration monitor. In embodiments where the wearable detector is communicating with the vibrational stimulator, the communication may include instructions to the stimulator unit related to frequency, power level, duration, or other relevant commands. In alternate embodiments, these instructions are transmitted to the vibrational stimulator from the processor of the external user device, e.g., a cell phone. Regardless of the source of communication, the stimulator can be of any configuration applicable to the location on the user's body and consists of a housing that will protect and position a vibrational element adjacent the vagus nerve, examples of which include an offset motor, piezo vibrator, or any device which rhythmically taps the patron with the intent of reducing pain or calming the patron like a mother does when she pats a baby. The housing also contains communication means for communicating with the IBI detector and user device. It should be noted that the ear is being described herein as the stimulation location of the vagus nerve, however any accessible location for placement of the vibratory stimulator able to provide stimulation to the nerve can be used.

The respiration monitor in most embodiments is a chest band capable of transmitting expiration data to the microprocessor within the IBI detector or the processor of the user device. Alternatively, a nasal implant/thermistor combination can be used having communication capabilities that communicate between the two elements. In most cases, a breathing app loaded on the external user device will guide the subject to paced breathing and there is no need to monitor it.

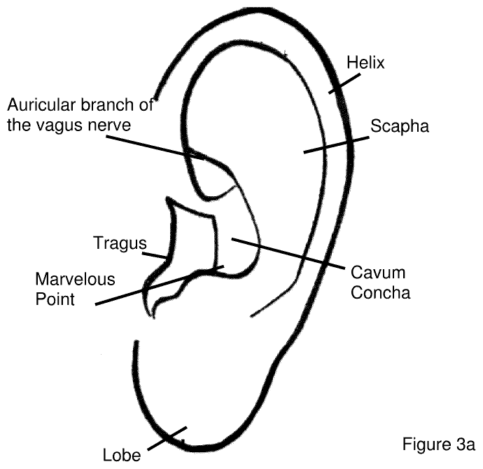

The vagus nerve is connected through branches to the vocal cords and the inner ear. The vagus nerve is known to be stimulated by singing, humming, yawning, and external sounds in addition to the electrical stimulation previously mentioned. Traditional Chinese medicine and Nogier auriculotherapy identify a particular location in the ear as a Vagus nerve stimulation point known as the Marvelous point, the location of which is shown in FIG. 3a. The vagal sensory innervation of the ear (known as the watershed zone of the auricular branch of the vagus nerve, shown is gray in FIG. 3a), contains a “Marvelous point”, is located in the cavum concha opposite the level of the external auditory meatus and tragus, which can be easily located anatomically. Based on acupuncture therapies, stimulation of the Marvelous point is believed to restore parasympathetic balance, lower blood pressure, and relieve muscle tension. Other effective locations would be classical acupuncture/Nogier points that mainly overlap with the vagus nerve watershed of the ear.

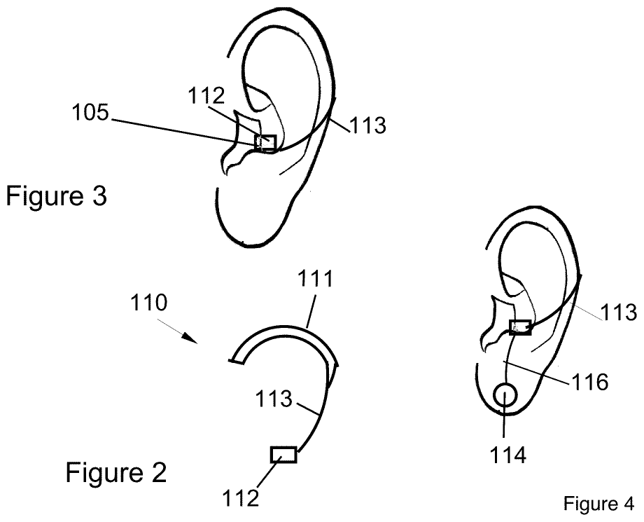

One example configuration of the ear stimulator unit 110 is illustrated in FIG. 2 having a housing 111 placed behind the ear, similar to a hearing aid, and positioning the stimulator unit 112 (see FIGS. 2, 3, 4) at the Marvelous point 105. The ear stimulator unit 110 also includes a stimulator microprocessor (not shown). The housing 111 and stimulator unit 112 are connected via wire 113. Although wireless connection between the two can be incorporated, the additional components will affect both size and weight. In the disclosed examples, vibrations are delivered to the watershed area of the vagus nerve endings in the concha of the left ear. While the vagus nerve is referred to as a single nerve, it is actually a pair or nerves that emerge from the left and right side of the brain stem. It is believed that vagus nerve stimulation of the right side may cause cardiac complications such as dysrhythmias since the right vagus nerve supplies the sinoatrial node. For this reason, the vibrations are preferably applied in the left ear, though application in the right ear may be feasible-depending on physician evaluation of the individual patient. When combined with the IBI detector 130 located in the wrist or thumb of a preferred embodiment, the electronics would be contained within the IBI detector 130, leaving only the microprocessor with communication means and stimulator 112 to be contained within the ear unit. The microprocessor receives information from either the IBI detector or the external user device with instructions for applying vibrations including initiating vibrations, strength of vibrations, frequency of vibrations, and duration of vibrations.

In an alternative embodiment of FIG. 4, the IBI detector is contained within an ear clip 114 that is connected to the stimulator 112. In this illustration, the ear clip 114 and stimulator unit 112 are connected through a wire 116 in order to reduce size and weight; however, the ear clip 114 can also contain communication means to enable wireless communication. In this example, the stimulator unit 112 is a highly accurate, heart rate variability monitor wherein the microprocessor processes the raw data received from the ear clip 114 which contains a PPG, IBI sensor, such as a photo-plethysmography (PPG) sensor having sufficient accuracy to measure the IBI. The microprocessor within stimulator unit 112 controls the decision when to engage stimulation as well as the duration, intensity, or frequency of the stimulation. The example illustrated herein applies the vibration stimulus to the water shed area in the concha of the ear in the area of the anatomically documented area of the known nerve endings of the auricular branch of the vagus nerve.

The stimulator 112 and ear clip 114 of FIG. 4 are connected to one another through wiring 116, Bluetooth, or other short range connection methods. The ear is in an inconspicuous location, however other areas of the body can also be used to apply vibrational stimulation on the vagus nerve as known in the medical arts. The incorporation of the IBI detector within the ear clip 114 is dependent upon the user and whether sufficient HR can be detected. This will vary from user to user and will determine the location of the IBI detector. When the IBI detector is placed within the ear clip 114, the microprocessor can be moved to the housing 111 of the stimulator 112.

In the examples set forth above, a chest band 120 may be used to monitor inspiration and expiration; however, a nose clip (not illustrated) connected to the stimulator 112 by a wire or other communication method can also be used. The IBI detector 130 uses the data received from the chest band 120, or other device, during exhalation for analysis as that is when there is greatest vagal activity. Other options for a respiration monitor would be incorporation into nose jewelry such as nose cuff, piercing, non-perforated U's, etc. In embodiments where a paced breathing app is incorporated, the need for measuring the respiration rate could be eliminated in many cases.

An alternate treatment method would be through use of a pen light device that would supply vibration at the ear working in conjunction with a thermistor attached to a nasal implant, like a snoring device. The thermistor would be used to monitor exhalation as well as to turn on and off the vibratory stimulator at the end of the penlight. Upon sensing a hot flash, a woman or man would put the canula in a nostril and hold the stimulator up to a specific point in the ear. A circuit within the vibrational device would take the thermistor input from the canula and gate the stimulator according to the breathing cycle. This action would shift heart rate variability power into the HF part of the spectrum and stop the hot flash. This method is a breathing guidance that signals when to exhale and use the vibrational device. In some embodiments communication with a smart phone can be incorporated to assist in the timing of breathing and monitor progress. Although a canula and thermistor can be substituted for the chest band, with a mechanical method of initiating the stimulator 112, the freedom provided by the use of the chest band is sacrificed.

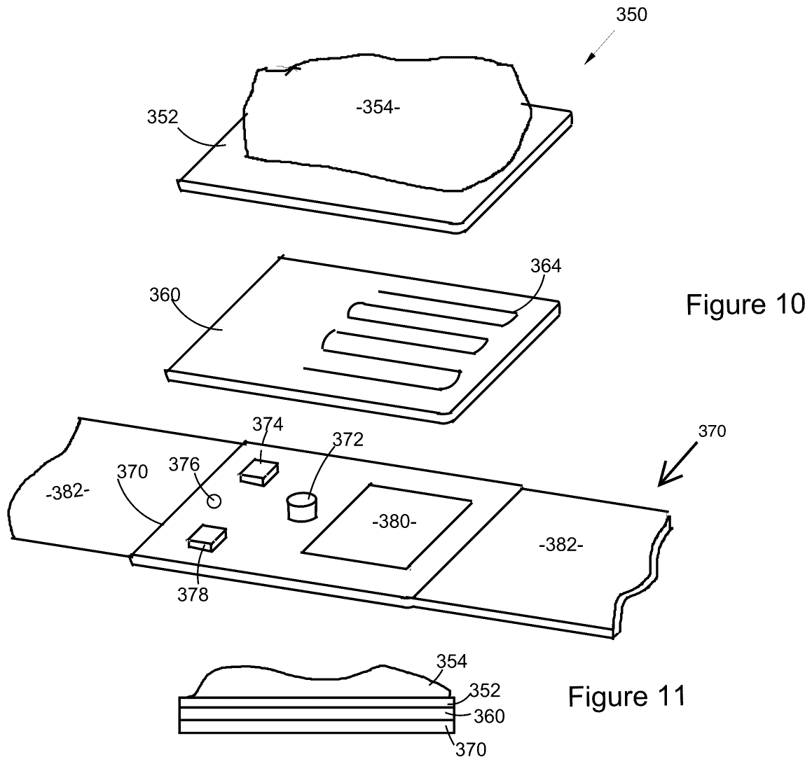

When the IBI detection devices are smaller in size, external securing members may be required for maintaining the IBI detector in position unless the substrates are extended beyond the device. The external securing members can include clamps, hook and loop, tape, etc. depending upon the time period of use. Additionally, when long term use, constant or sporadic, is predicted a more permanent method, such as incorporating the securing member, as described in FIG. 10, as part of the IBI device substrate can be used.

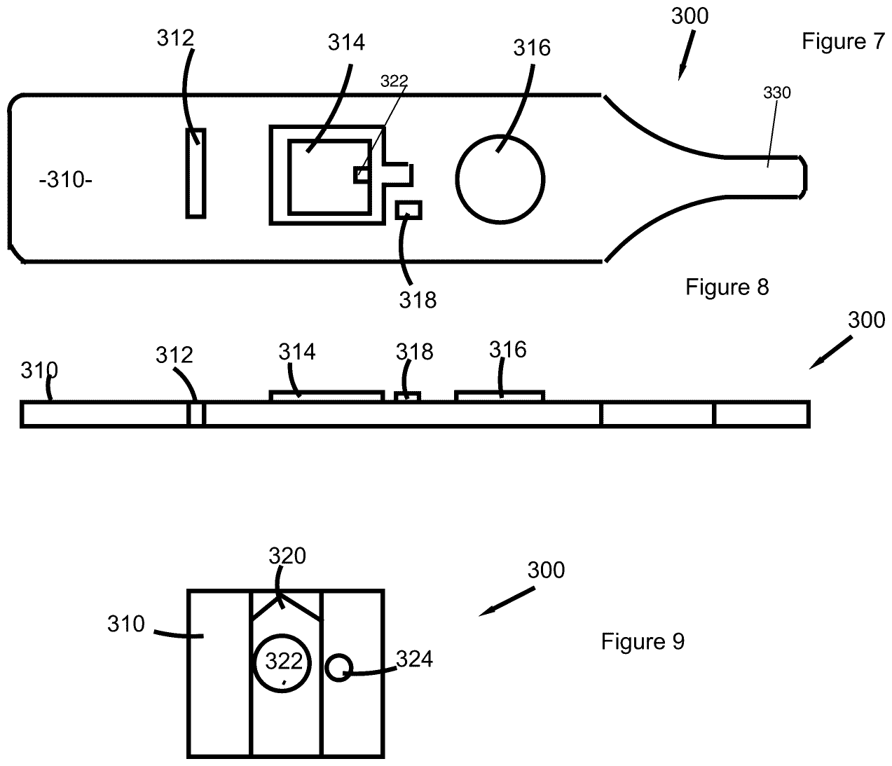

Another embodiment of a finger cuff is illustrated in FIGS. 7-9, wherein the pressure sensor 322 is welded into the bladder substrate 314 and sealed with a 1 PSI air release, preventing leakage of trapped air under ordinary pressure. The microprocessor 318, as with the other disclosed embodiments, controls the functions and communication of the cuff 300.

The microprocessor 318 is connected to the battery 316 and pressure sensor 322 by wires, traces or other means depending on material of manufacture. The microprocessor 318, as with other embodiments, communicates with the stimulator 112 and any smart devices by appropriate methods. The pressure sensor 322 also connects with the LED 324 to assist with monitoring pressure.

To initiate application the tab 330 of the substrate 310 is threaded through the slot 312 at the opposing end of the substrate 310 and tightened. A clamp 320 having the ability to be tightened through screw 322 is placed around the cuff 300 and tightened until the LED 324 indicates that sufficient pressure has been built within the bladder 314. An example of a clamp 320 would be a hose clamp or a releasable zip tie.

FIGS. 10 and 11 illustrate an alternate IBI detection device 350 that has the option of being waterproofed through the enclosure of the assembled IBI detection device 350 in polyurethane or its equivalent. The contact substrate 352, charging substrate 360 and electronics substrate 370 are manufactured from a semi-rigid material compatible with a plastic weld such as JB Weld, Loctite, etc. The substrates can be molded, extruded, printed, or manufactured in any other method applicable to the material used. The substrate is manufactured from a universal safe material for skin contact such polyurethane or its equivalent. Flexible circuits will reduce the electrical parts to about a mm in height, less if IC packages are eliminated for direct die bonding to a flexible substrate.

In the embodiments where a bubble is being used to indicate the blood pressure, a pump is not required. The bubble is placed adjacent the monitoring location with the blood vessel expansion being, as indicated heretofore, registered by a pressure sensor hermetically sealed in a volume of gas. Any of the bubble IBI detection devices can be sealed for waterproofing and in some embodiments the battery is rechargeable.

The contact substrate 352 contains the bladder, or in this example a bubble 354, preferably manufactured from a two-mill polyurethane or its equivalent, secured to the contact substrate 352. The bubble 354 can be plastic welded to the contact substrate 352 or, in some applications where beneficial, the contact substrate can be manufactured with a top and bottom substrates and the bubble 354 secured between the two when secured together.

The illustrated IBI detection device 350 uses a piezoresistive pressure sensor, or its equivalent. In most uses, all elements within the finger cuffs should have a thickness less than a millimeter although sizing will depend upon end use.

The illustrated IBI detection device 350 uses a piezoresistive pressure sensor, or its equivalent. In most uses, all elements within the finger cuffs should have a thickness less than a millimeter although sizing will depend upon end use.

The charging substrate 360 includes the wiring for the inductive antenna 364 to enable recharging of the battery 380. Due to the size of the IBI detection device 350 the material of manufacture for the antenna 364 should be kept as minimal as possible and lightweight materials such as aluminum, copper, brass, etc. should be used, although the weight of the antenna will be a few grams. Optimal antenna materials for use with the material of substrate manufacture will be known to those skilled in the art. Alternatives would be to incorporate a USB port or to replace the battery, however all elements would not be sealed onto the bladder and waterproofing would be compromised or eliminated.

The electronics substrate 370 contains the battery 380, microprocessor 378, LED 376 and Bluetooth radio 374 in addition to the pressure sensor 372. The electronics can be placed onto any appropriate substrates, such as a flexible circuit board, in methods appropriate to the substrate. Preferably the finger cuffs disclosed herein should be approximately the size and thickness of a band-aid.

Placement of the battery 380 on the electronics substrate must be such that it aligns with the inductive antenna 364 if the recharging feature is included. This method of recharging is well known in the electronics arts and placement, antenna size, etc., will be known to those skilled in the arts. In the event the IBI design is such that battery 380 is to be replaced or a single use, the placement of the antenna 364 and battery 380 are not critical. The Bluetooth radio 374 forwards information received by the microprocessor 378 to the chosen stimulus device.

As noted heretofore the LED 376 indicates that the sufficient pressure has obtained based on information received from the pressure sensor 322. As the IBI detection device 350 will be welded along the edges of the substrates, the elements within each substrate must be spaced from the end a sufficient distance to prevent obstruction of the element's function. This is especially true of the electronics substrate 370 and charging substrate 360.

Illustrated in this embodiment the electronics substrate 370 has been extended to incorporate a wrap 382 that can be used to secure the IBI detection device 350. The wrap 382 can be secured by hook and loop (not illustrated) or other means compatible with the material of manufacture. As the thickness of the IBI's disclosed herein is minimal, a removable tie or clamp, as noted above, can be used.

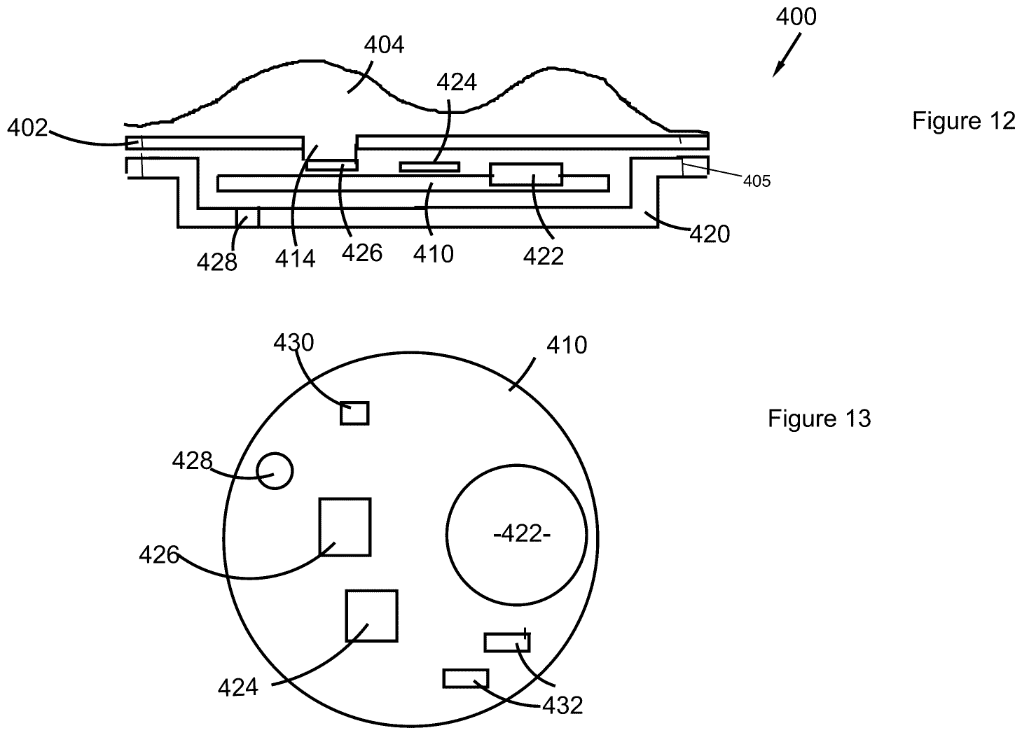

Another embodiment of the small, sealed, waterproof IBI detector is illustrated in FIGS. 12 and 13. This embodiment uses a gauge pressure sensor 426, thereby requiring an air passage 414 within the contact substrate 402 to enable air from the bubble 404 to provide additional force to the sensor 426. The contact substrate 402 and the bubble 404 are welded along 405 to secure the contact substrate 402 to the base substrate 420. Secured between the contact substrate 402 and base substrate 420 is the circuit board 410.

The circuit board 410 contains a battery 422 with charging pads 432. The charging pads 432 would be used in embodiments where the battery 422 is rechargeable and can be USB ports or other applicable recharging methods. Alternatively, the battery 422 can be small and non-rechargeable for single or limited use. In embodiments where the battery 422 is replaceable the method of securing the contact substrate 402 and the base substrate 420 would need to such that the two elements could be reconnected without damage to the IBI detection device 400.

The microprocessor 424 contains communication means, as previously described, to transmit data to the selected analyzer and/or a smart device.

In this embodiment an on/off switch 430 is provided to save battery 422 power. When the battery 422 is non-rechargeable, this feature would be eliminated. The physical switch to the on/off switch 430 is not illustrated but would extend through the base substrate 420 for external access. Preferably the LED 428 remains on the circuit board 410 side of the base substrate 420 and the IBI detection device 400 sealed with translucent polyethylene, or its equivalent. Alternatively, the physical LED 428 could extend through the electronics substrate 410 to be visible through channel 428.

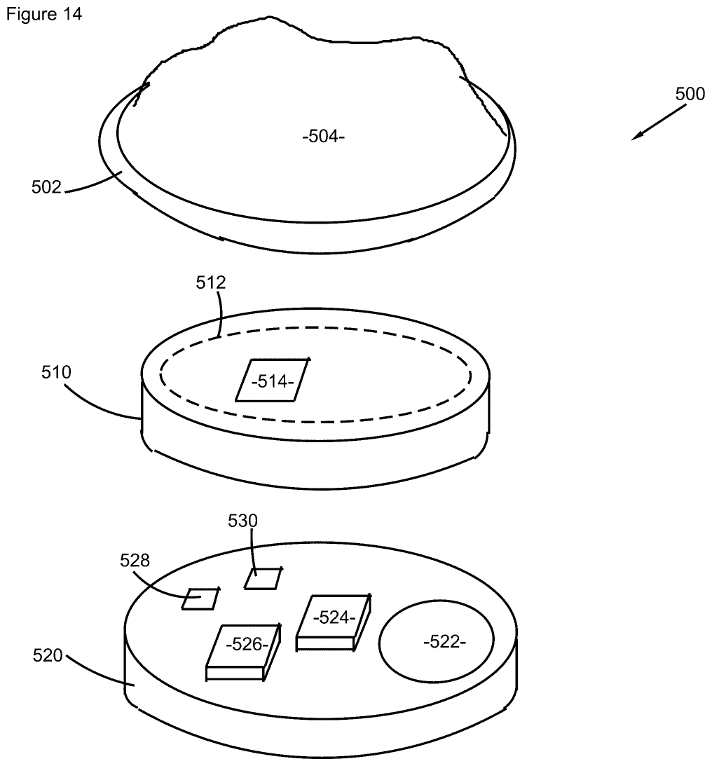

FIG. 14 illustrates another embodiment in IBI detector 500. In this embodiment the welding edge 502 is illustrated to enable the bubble 504 to be welded to the substrate 510 at weld line 512. It should be noted that this bubble 504 design can be incorporated in all of the foregoing embodiments. To provide additional pressure to the pressure sensor 526, a channel 514 is placed in the substrate 510, over the pressure sensor 526. The microprocessor 524, as noted in the foregoing embodiments, contains communication means in order to send data to the stimulator and any desired smart devices. As with the IBI detector of FIGS. 12 and 13, an on/off switch 530 and LED 528 are also incorporated and embedded within the base substrate 520.

As the size of the foregoing finger cuffs does not permit an incorporation of a sphygmomanometer or relative blood pressure sensor as the proximal phalanx cannot be squeezed to stop the circulation without considerable discomfort or pain, the finger cuffs may require periodic calibration using, for instance, a Bluetooth enabled upper arm cuff or other absolute blood pressure measurement device. The finger cuffs provides relative beat by beat blood pressure without calibration. However, a number of physiological parameters, beyond those indicated above, can be determined and are comparable to those provided by VitalStream, many without calibration.

### Method of Analysis

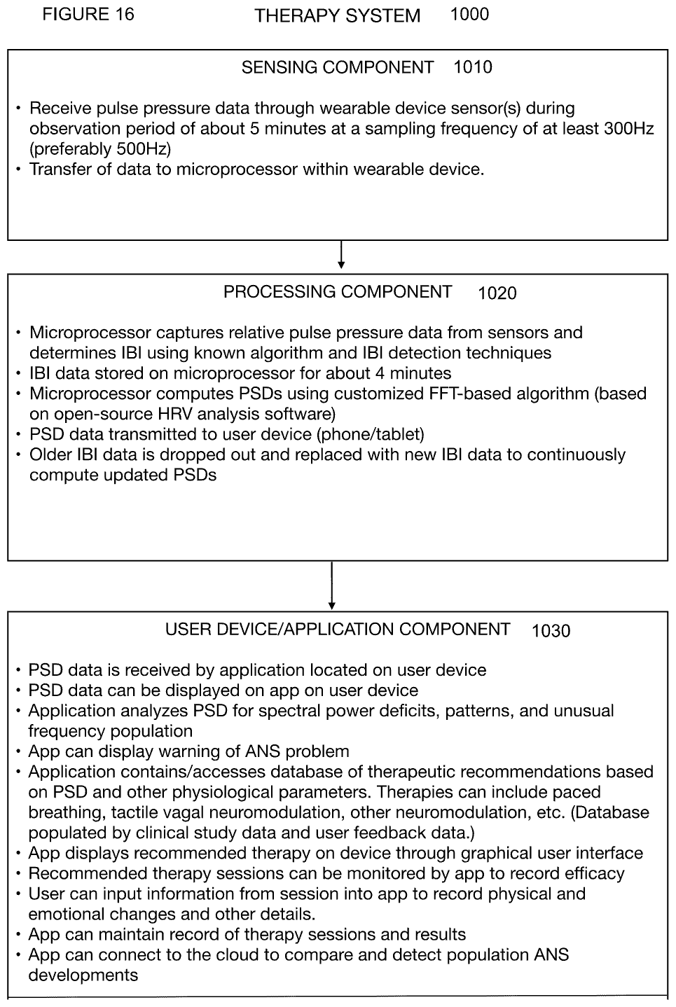

Power spectral density (PSD) is a plot of the activity of the autonomic nervous system (ANS). Imbalance or depressions as observed in the ANS are associated with many symptoms of ANS dysfunction. Neurostimulation actions, including paced breathing and vibrational stimulation, can train or strengthen all or parts of the ANS resulting in physiological and psychological changes as revealed in the PSD. The wearable detector of the present invention as described above is used in conjunction with a user device such as a mobile phone or tablet running computer application to receive and analyze the data collected from the wearable detector in order to determine ANS status based on PSD and provide both responsive and preventive therapies. The algorithm of the computer application notes the dysfunction, recommends a therapy, tracks the progress of the therapy, and makes decisions to possible changes in the therapy. The algorithm will contain all known therapies and will be constantly supplemented with new therapies as they become available to lead to better health outcomes in treatments. As shown in FIG. 16, the therapy system 1000 of the present invention includes a sensing component 1010, a processing component 1020, and an application component 1030

Power spectral density (PSD) is a plot of the activity of the autonomic nervous system (ANS). Imbalance or depressions as observed in the ANS are associated with many symptoms of ANS dysfunction. Neurostimulation actions, including paced breathing and vibrational stimulation, can train or strengthen all or parts of the ANS resulting in physiological and psychological changes as revealed in the PSD. The wearable detector of the present invention as described above is used in conjunction with a user device such as a mobile phone or tablet running computer application to receive and analyze the data collected from the wearable detector in order to determine ANS status based on PSD and provide both responsive and preventive therapies. The algorithm of the computer application notes the dysfunction, recommends a therapy, tracks the progress of the therapy, and makes decisions to possible changes in the therapy. The algorithm will contain all known therapies and will be constantly supplemented with new therapies as they become available to lead to better health outcomes in treatments. As shown in FIG. 16, the therapy system 1000 of the present invention includes a sensing component 1010, a processing component 1020, and an application component 1030

The sensing component 1010 involves one or more sensors of the wearable detector reading pressure pulse data and transferring the data to the wearable detector microprocessor. The processing component 1020 involves the microprocessor detecting IBI through known IBI-detection techniques using available software for digitizing real-time pulse pressure signals and extracting the IBI for each successive heartbeat. The microprocessor further extracts all common time domain parameters of the HRV. IBIs are obtained through an observation period of approximately 3-5 minutes where time resolution and sampling frequency is measured above 300 Hz, and preferably near 500 Hz. The microprocessor is continuously computing new IBI data which is stored on the microprocessor for a short period of time, preferably around four minutes, after which time the oldest IBI data is dropped, thus allowing for continuously updated PSD calculations. IBI data is used to compute PSDs using a customized FFT-based algorithm based on open-source HRV analysis software. The PSDs must be developed in a format compatible with mobile communications devices so that the PSD data can be transmitted to a user device such as a mobile phone or tablet.

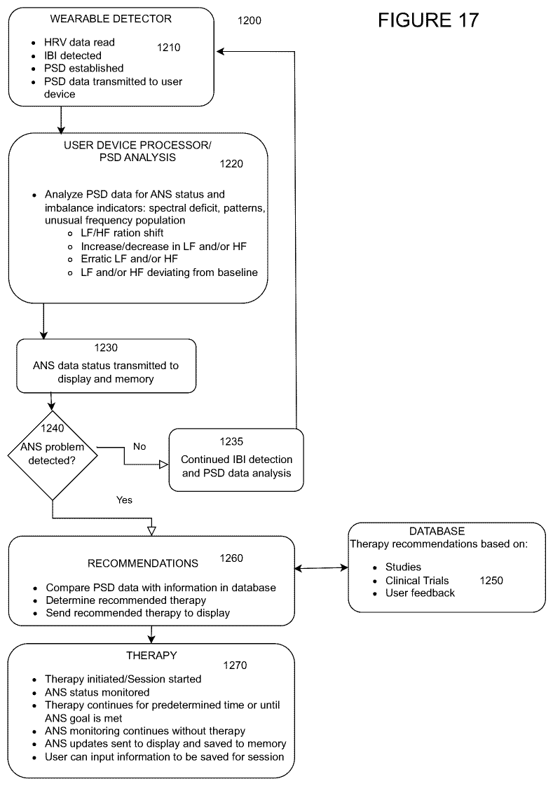

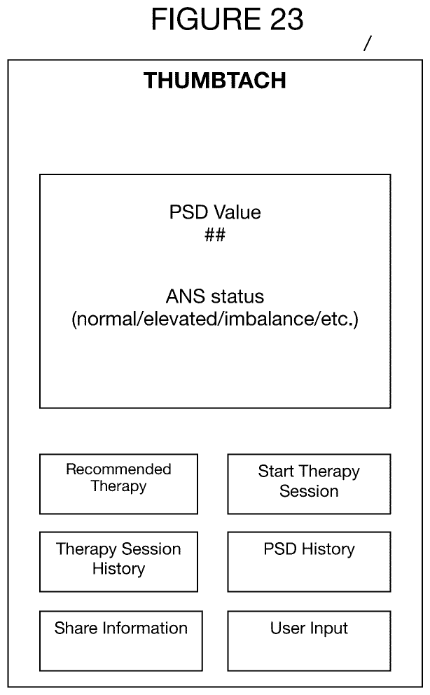

The application component 1030 of the present invention involves using a computer application to analyze the PSD data transmitted from the wearable detector to the user device. The user device such as a cell phone or tablet includes at least a processor, a display, a database, and memory. An example graphical user interface 1100 of the display for the computer application is shown in FIG. 23. As shown in FIG. 17, software algorithm 1200 of the present invention analyzes PSD data to detect and correct ANS dysfunction. The wearable detector 1210 computes and transmits PSD data to the processor of the user device 1220 which analyzes PSD data to determine PSD value and monitor ANS status and transmit the information 1230 to the display and memory of the user device. ANS status changes with the detection of ANS imbalance indicators including but not limited to spectral power deficit, patterns, and unusual frequency population. Power deficits include increases or decreases in LF or HF which in turn alter the LF/HF ratio indicating an LF/HF imbalance. There is no “normal” LF/HF ratio thus a baseline ratio and regular patterns are established for the individual user. Noticeable patterns can include observed movement of LF and/or HF prior to symptoms such as a recurring drop in HF prior to hot flash onset.

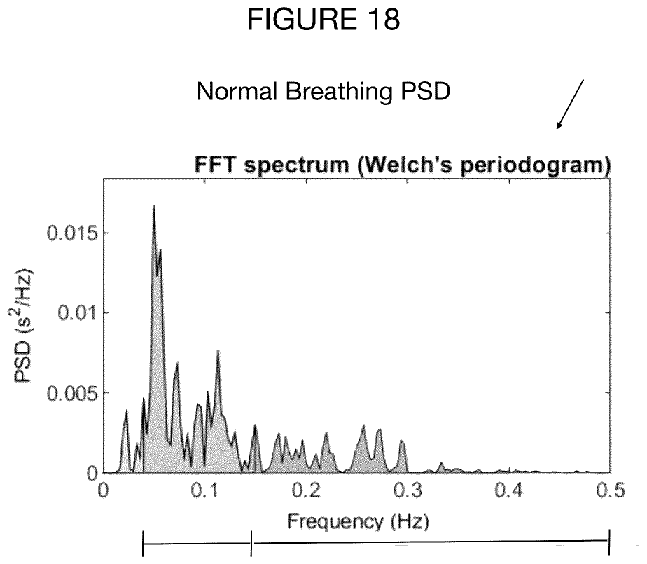

Unusual frequency population can include erratic HF and/or LF readings or LF and/or HF readings that deviate from commonly normal readings or individual baseline readings. As shown in the graph 1300 of FIGS. 18, typical PSD readings for individual at a normal breathing rate include an LF 1310 in the range of 0.001-0.01 s2/Hz and an HF 1320 in the range of 0.0-0.004 s2/Hz. As long as there are no ANS imbalance indicators, the device of the present system continues to detect IBI and compute and analyze PSD 1235 while transmitting the PSD value 1110 ANS status 1120 to the display as shown in FIG. 23.

It has been determined that shifting the PSD to collapse HF and LF is beneficial to relieving ANS imbalance symptoms. Further, studies and clinical trials suggest therapies that work to shift the PSD in a desirable way. Such recommended therapies are one of or a combination of techniques including as paced breathing, vagal stimulation, and other neuromodulators. When the PSD analysis of the present invention indicates an ANS imbalance 1240, an imbalance notification is displayed, and the PSD data is compared with information in the database 1250 to determine a recommended therapy 1260. This recommended therapy is then displayed on the user device 1130 as illustrated in FIG. 23. The user can start a therapy session 1270 on the device 1140 and guide themselves through the recommended therapy during which the wearable detector continues to detect IBI and compute PSD of analysis by the application. Shifts in the ANS status and related physiological parameters such as time HRV parameters, heart rate, and relative blood pressure are recorded by the application and can be both stored in memory for future reference and accessed through the computer application through features such as therapy history 1150 and PSD history 1160 and shared 1170 with the database for incorporation into therapy data. Further, the user can input 1180 information into the application such as physical changes, emotions, etc. experienced during the therapy. Further, the application can connect to the cloud to compare and detect population ANS developments, outbreaks of illness, and anxiety levels. ANS status records and therapy session records can be shared with healthcare providers.

### Method of Use

The system of the present invention monitors the user's ANS state and provides suggested therapies for treating symptoms and/or strengthening the ANS. These therapies can include one of or a combination of techniques including paced breathing, vagal nerve stimulation, and other neuromodulators.

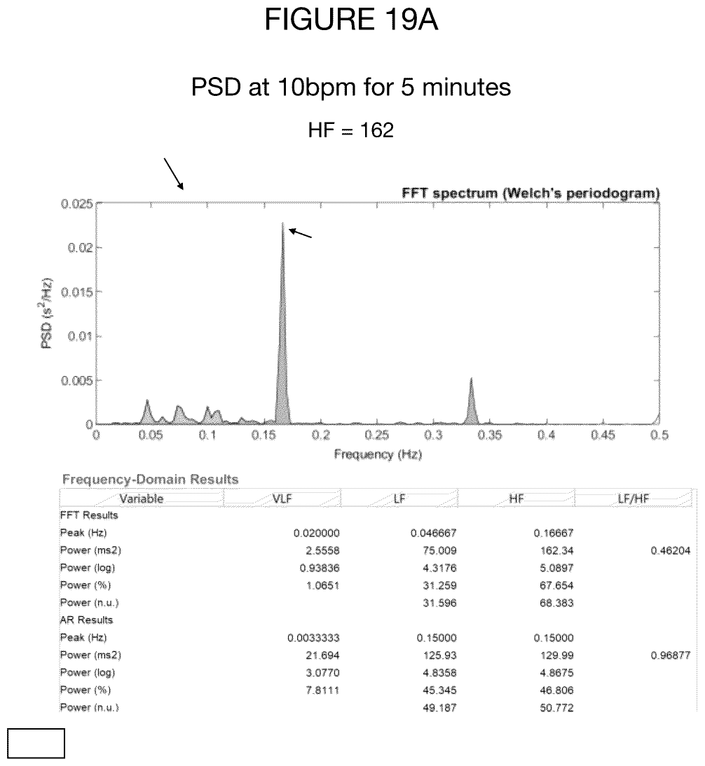

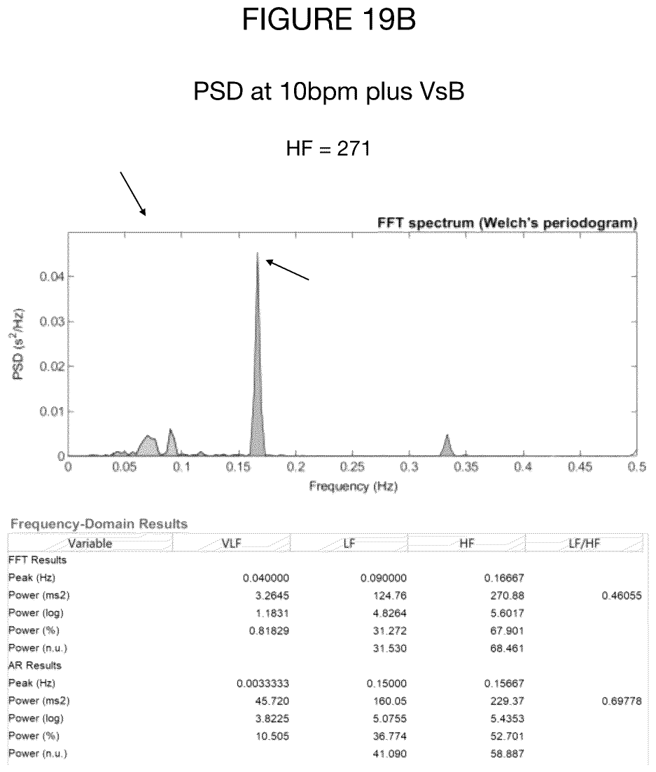

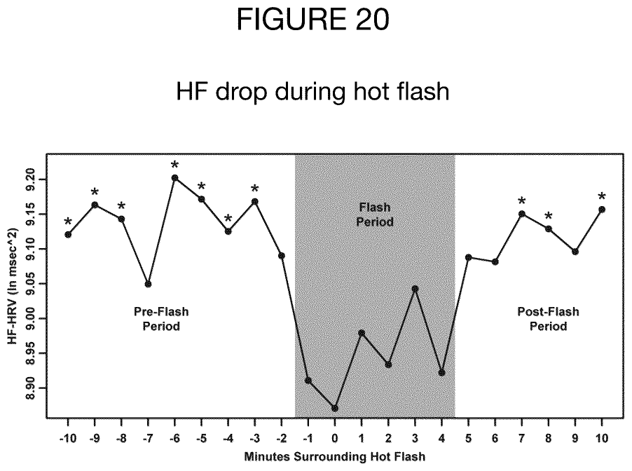

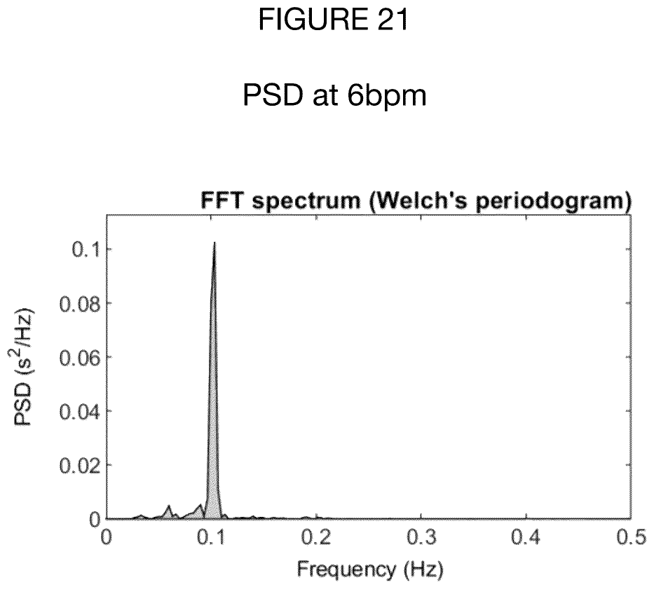

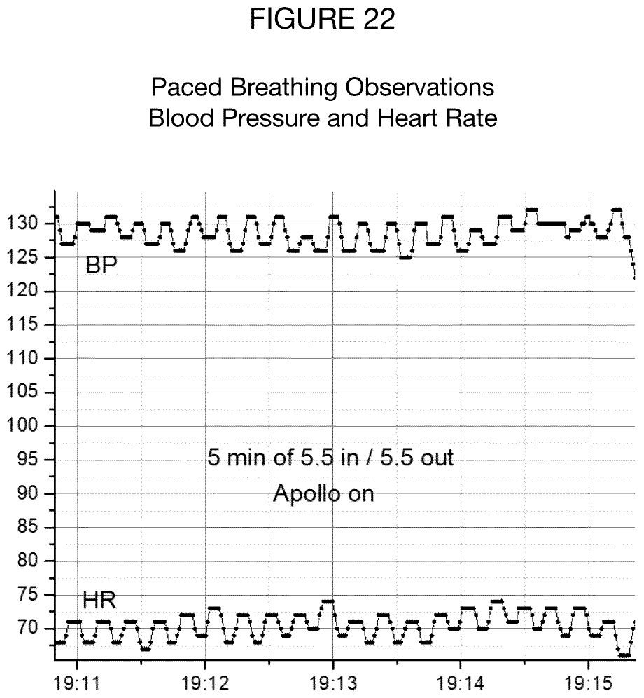

Optimal results are often observed when paced breathing is combined with vagal nerve stimulation during an exhale. As shown in the graphs 1400 and 1500 of FIGS. 19a and 19b respectively, paced breathing practice of 10 beats per minute strengthens the collapse of PSD 1410 to about 0.02-0.025 at 0.167 Hz for an HF of 162 ms2, while paced breathing at 10 bpm plus vagal nerve stimulation strengthens the collapse of PSD 1510 to over 0.04 at 0.167 Hz for an HF of 271 ms2. While paced breathing alone and nerve stimulation alone shift the ANS, paced breathing plus vagal nerve stimulation produce a great shift of ANS within the HF range. As disclosed herein, by stimulating the vagus nerve through vibration during exhalation and peak HF spectrum, a shift in heart rate variability power occurs thus allowing for a rebalance of the HF/LF ratio.

Mechanical vibrations at the ear during exhalation have been shown to result in a distinct peak in the HF spectrum indicating all the power is concentrated at the optimal frequency. The disclosed system uses vibration (VsB) to the ear to produce parasympathetic nerve stimulation to lower LF/HF ratios thus providing a number of health benefits including minimizing or eliminating hot flashes, anxiety, etc. While applying vibration to the ear is specifically referenced herein, the vibration can be applied at other locations beside the vagal watershed, as known in the medical arts, that would stimulate the PNS resulting in a peak in the HF spectrum. This is achieved mainly through stimulation of the vagus nerve using the disclosed low-cost, highly accurate mechanical vibration system during exhalation rather than through prior art electrical stimulation.

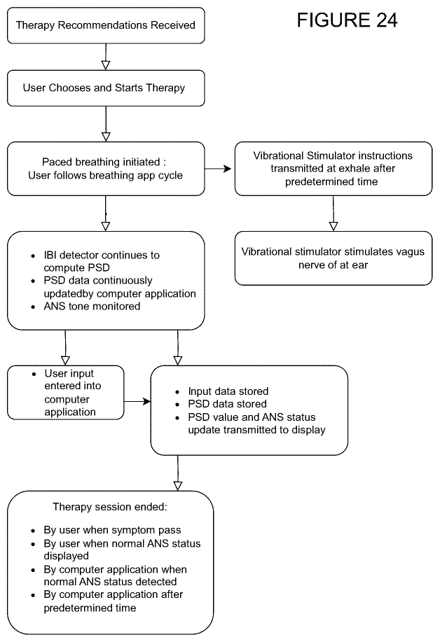

When a user of the present invention attaches the wearable detector described above, obtains PSD analysis with ANS imbalance indicators, and receives recommended therapy, the user can self-guide themselves through the chosen technique an example of which is illustrated in FIG. 24. For instance, the user may receive a recommendation to breath at 10 bpm for 5 minutes and then apply vagal stimulation during an exhale. Preferably, the user is guided through paced breathing by the computer application and vagal stimulation is prompted through the computer application in wireless communication with the vagus nerve stimulator at a predetermined point in a breath cycle. Alternatively, the stimulator can be prompted through an external controller after being prompted by the breathing application. While the user is practicing the therapy, the IBI detector continues to capture data, compute PSD, and transmit data to the computer application where the updated PSD/ANS status is continuously displayed. If a respiration detector is being used, respiration data can be combined with PSD data to automatically prompt vagal stimulation at the preferred time during the paced breathing cycle. The user can continue the therapy for a period of time recommended or until symptoms cease. Information from the therapy session as well as user feedback is saved to the application. Optimal corrective therapy involves training. A novice is less in need of the labor currently required during personalized instruction to reach a goal.

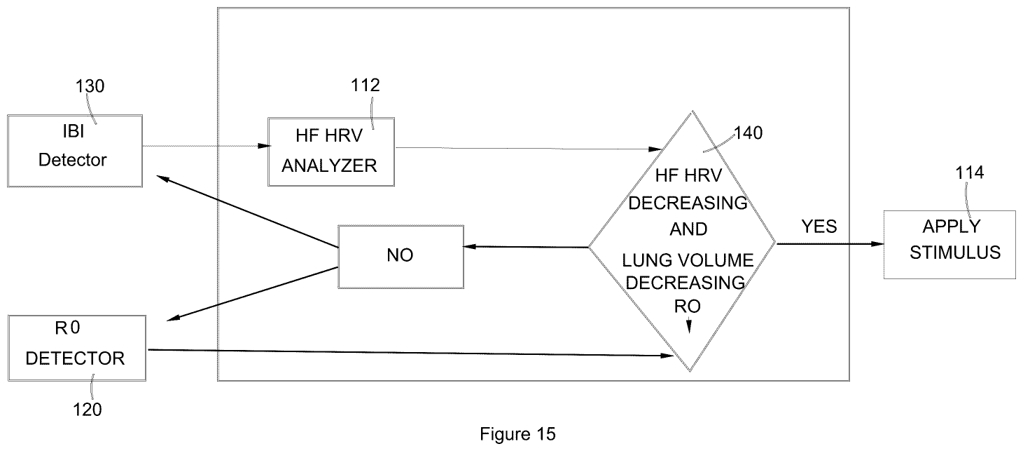

FIG. 15 illustrates a flow chart of a particular embodiment involving the data transfer between the IBI detector 130 and respiration detector 120, if a breathing app is not used, and its analysis at microprocessor 140 within the HRV analyzer 112. Data from both the IBI detector 130 and respiration detector 120 are sent to the HF HRV stimulator 112 where it is received by the microprocessor 140 for analysis. If the data meets the criteria of the HRV, as set by physiologists, showing a decrease in HF HRV and respiration rate, vibrational stimulus is applied. Respiration is monitored to apply the vibrational stimulus during exhalation for prime vagal activity. To provide the highest accuracy, the HRV PS is analyzed every heartbeat in a rolling method where the oldest or first heartbeat is dropped from a five-minute observation and the newest beat or most recent is added. Results are as real-time as possible.

After using the device for a period of time and developing a user history with populated ANS data and session data, the computer application can suggest ANS strengthening training sessions and thus begin to prevent some ANS symptoms as well as treating active issues. For example, with more uses of the device, a menopausal user can develop a record of when and how often hot flashes occur, recurring PSD data prior to hot flashes, and the most effective therapy, all of which can be used to individualize both responsive treatment and preventative ANS training. Although an optimal PSD is known and is achievable by meditators, it is not easily accessible by untrained meditators. The disclosed system provides an apparatus and system that enable users to monitor their PSD and assist in bringing the user to the 0.1 Hz level or other desired frequency through vibration at the vagus nerve.

As noted above, the disclosed system is applicable to more than just hot flashes, which generally do not require medical intervention or recording, and the ability to be wirelessly connected to a smart device to alert caregivers, update patient records, or record data for large studies can be beneficial in some instances. The IBI detector 130 sends the PSD data to the smart device which then analyzes and tracks the data received against predetermined parameters and adds current information to the database. Where ANS intervention is required, therapy is completed and the results are recorded and saved. These saved therapy session can be shared with caregivers giving them insight into the frequency and severity of ANS issues, efficacy of treatments, and user feedback. Since the system disclosed herein is capable of monitoring progressive changes in blood volume and changes in blood pressure, in addition to the previously described functions, the system can alert the user of any medical issues that can benefit by the stimulation of the vagus nerve such as pain, PTSD, depression, insomnia, heart problems, narcotic withdrawals, anxiety and stress.

The transmission of data between smart devices and IBI detectors, such as Fitbit, is well known in the art and can be incorporated within the devices disclosed herein. As the smart device is in communication with the microprocessor within the IBI detector, or incorporated therein, the smart device can also make recommendations for or orchestrate a procedure to correct a fault in the power spectral distribution using one or more known methods of neurostimulation. Changes to the location of physical components within the system can also be made from a smart device.

### Example Applications

### Hot Flashes

Menopausal vasomotor symptoms, commonly known as hot flashes, in women undergo a drop in HF during the time of the hot flash as shown by Thurston et al and as illustrated in FIG. 17. Thurston et al also found that hot flashes are caused in part by parasympathetic (PSN) decline. This graph shows how the total power in the parasympathetic nervous system declines over a few minutes before and recovers as the symptoms recede. In addition to the discomfort of hot flashes, more serious menopausal problems include osteoporosis and cardiovascular disease. Men receiving androgen suppression therapy for prostate cancer treatments have also shown the same drop in HF.

It is known that various sensory inputs can be used to induce coherence whose observation is one aspect of this application. We believe that vibratory stimulation can add to or replace completely the need for paced breathing. One advantage of not requiring a conscious effort to breathe at slow and regular intervals is that that the treatment could be made to require no user input. At its simplest, patients with low LF would be directed into a set of therapies known to boost their LF and patients with a low HF would similarly be boosted to HF using HF training.

The disclosed invention treats hot flashes by detecting PNS loss in HRV. An intervention with ear vibration during paced exhalation at 10 breath cycles per minute appears to restore this loss. The preferred amount of intervention is being studied but may be quite small based on observations with ear vibration done for only two days and PNS effect. A subject with VMSs who used a defived paced breathing method (10 breaths per minute) with daily ear vibration to Marvelous point and showed increased PSDs in HF, lower LF, and felt less symptoms but had no sleep data recorded.

### Meditation

Slow, paced breathing at 6 breaths per minute produces an oscillation in the blood pressure in most people of about 0.1 Hz. The heart rate will also modulate at the same frequency but out of phase.

The disclosed system can be used as a meter to manually gate the vibrator during a specific phase of the breathing cycle, or the respiration cycle can be detected using a sensor of respiratory phase. Using the disclosed device, the user can monitor the physiological responses during meditation to assist in achieving the desired level.

In both cases, real-time heart rate variability analysis will detect a hot flash and automatically apply vibration. Here, keeping with the European standard requiring a five-minute observation window, real-time HRV analysis would use a running average over this time period or less. In this example, analysis is done every second on the last five minutes with the system discarding the first variance within the five minute monitoring and adding the new variance at the end. In this way, a moving picture is provided that is continuous and current, similar to a rolling average.

As disclosed in a paper by Kevin J. Tracey, MD, VsB, reduces inflammatory cytokines in rheumatoid arthritis (RA). “The Inflammatory Reflex,” Nature. 2002 December; 420(6917)853-9.doi: 10.1038/nture01321 The disclosed VsB routine may also work for bone disease since the same cytokine network is elevated in millions of women with osteoporosis that is a troublesome consequence of aging menopausal women and in men with prostate cancer.

Further, VsB can be used as a tonic for men and women with gender hormone withdrawal related cardiovascular problems including stroke, heart attack, thrombosis, lipid dysfunction, as a possible chronic treatment intervention or as an adjunct to drug therapy for this population.

### Pain Management

It is known that pain depends partly on blood pressure and when the blood pressure is high, pain is attenuated.

Part of pain can be lowered by forcing the body into LFOs. Forcing a single LFO in the blood pressure will greatly dominate the collection of all oscillations while the high frequency oscillations (0.15 to 4 Hz, HFOs) will greatly attenuate. At least one component of pain lowering is due to psychological perception because, when there is forcing and using the disclosed apparatus and method, the autonomic nervous system (ANS) transitions from a mix of parasympathetic and sympathetic nervous system oscillatory rates to one frequency of mostly all parasympathetic, which generally is accompanied by a calming effect.

It is known that training by applying a stimulus gated on the heart pulse lessens pain. Here, the two previous adjacent pulse rates are quickly averaged to predict the systole portion of the next future pulse. The prediction is poor because the heart rate is random and unpredictable, the method described here, because of forced coherence, makes the prediction of the next beat accurate and could lessen the chronic pain of patients with fibromyalgia by using a gated stimulus.

### Insomnia, Anxiety, Depression

The disclosed neurological stimulation system (NSS) is used to provide timing for gating another stimulating device for instance during a high blood pressure time period within the cycle of pressure oscillations itself or vibrations of longer periods, like six seconds This approach of using the blue spike to concentrate ANS power to where it is needed may work for a number of other chronic conditions like insomnia, depression. As an example, using this system the PSD of an asthmatic can be observed and monitored to see if the asthma is accompanied by a deficit in the PSD, say between two frequencies. If there is a deficit, like what occurs during a hot flash, then the device would recommend a therapy which would consist of some neurostimulation method which could be used for immediate freedom from symptoms or as a medication used to obtain future resilience to a stressor.

In addition to the health issues mentioned heretofore the disclosed system can be advantageous in monitoring inflammatory bowel disease, diabetes mellitus; suppression of opioid withdrawal systems and dementia prevention with tonification of the ANS.

### Oscillation Frequency and Respiration Information

Paced breathing forces the blood pressure into a state of pressure oscillation variation where the blood pressure oscillation frequency can be adjusted using this method and apparatus from around 0.05 to 0.40 Hz. These pressure oscillations are observed in the systolic blood pressure to be at the same respective frequencies as those observed in the heart rate and are dubbed low frequencies oscillations (LFOs). In systolic blood pressure, for instance, LFOs have been observed to be from 5 to 20 mmHg peak to peak amplitude about a narrow band of frequencies with characteristics similar to a classical resonance or spectral line as in optical or acoustic spectroscopy. Observation of a low frequency oscillation in the PSD is accompanied by the physical observation of an oscillating arterial system. Arterial pressure measurement devices are used to detect LFOs. As there is energy in oscillations and since this energy is distributed and stored within the oscillating arterial/brain system, energy can be added into this resonance with each correct breath cycle. Paced breathing can pump up this LFO vibratory phenomenon. The motion of the lungs generates relatively large amounts of infrasound vibratory energy that, at the correct frequency, may couple with the circulatory system's natural resonance. A variety of devices may add to this energy via physical stimulation or neuromodulation or be sufficient by themselves.

When various breathing rates are evaluated for their ability to result in BP and HR oscillations, the excitor frequency must be close to the oscillating frequency. This phenomenon behaves like a resonator, a system that can store energy and has a characteristic oscillation frequency.

In coupled harmonic oscillators, the coupling is particularly good if the frequency of the driver is the same as that of the resonant system. Efficiency suffers when the frequencies do not match. One common human LFO is considered to be 0.1 Hz with lower LFOs often observed in older people and elevated LFOs often observed in younger people. Most studies use 0.1 Hz for modulation including physical, neurological, optical, tactile, auditory, electrical, and a variety of devices that the body can sense.

There are many methods which will force the oscillation frequency to shift to higher or lower frequencies, the simplest is slow, paced, abdominal breathing. The Apollo Neuroscience device appears to force an LFO in test subjects at 0.1 Hz, where the device is resonately turned, and it is an easy method to produce an oscillation that will persist for many minutes. By changing the respiration frequency within certain bounds, the oscillation resonance frequency will be changed. Thus, breathing rate, for instance, can be used to tune a resonance, and it is expected that neurostimulators, when exhibiting a frequency of application at the desired resonance frequency will result in expression of the desired resonance at the same frequency.

Single frequency oscillation is an indication of coherence which can be thought of as a harmonic relationship between the respiration rate and the heart rate, like two notes played in the same musical key. Blood pressure oscillations with a frequency near 0.1 Hz are historically called Mayer waves.

Gating has been demonstrated on knowing the respiration cycle, which exhibits the respiratory sinus arrythmia (RSA) so that times within the cycle are used to gate a stimulator. This method requires the patients to be trained to breathe at 0.1 Hz (six breaths per minute), which is a powerful forcing simulator for single frequency LFO generation. By knowing the respiration cycle times, high blood pressure times or any periodic time in the cardiac cycle can be observed and then used to gate a remote stimulation device.

The disclosed system can force the body into a single HFO, where times of high blood pressure within the cycle period of the LFO can be used to gait a remote stimulator. Initially monitoring will be required to observe the oscillatory nature of the

The oscillatory nature of BP using a device such as VitalStream or another beat-by-beat blood pressure measuring device can be used to confirm that a single line in the PSD causes the BP to oscillate at the same frequency. Once the phase of the LFO is determined it is then possible to predict the times that the blood pressure is high, which establishes the times needed to gate the remote stimulator. Both the stimulator forcer and the stimulator for synchronous pain reduction training can be the same device and noninvasively mounted on the ear where there is direct access to the vagus nerve.

The foregoing is a description of cardiac gating or gating within the cardiac cycle. The method must apply a stimulus at a precise time during each heart cycle.

However, it is impossible to predict the arrival time of the next heart cycle because of HRV. By imposing an oscillation on the heart rate, the arrival time of the next beat is predictable because the oscillation is a sine wave. Then, a stimulus may reliably be applied at the correct time in the cardiac cycle. The sign wave is fixed by either paced breathing or stimulator. The frequency of the oscillation in heart rate and also identified in the PSD is the same frequency. If the HR known at any point along the sine wave, see FIG. 16, this fiducial point will next occur in the HR of the next beat can be predicted.

In some, there may be robust LFOs in the heart rate, which can be used to time the gating of the remote stimulator.

### Vagus Nerve (VN)—Respiration Relationship Information

It is known that respiration modulates VN activity, namely by suppressing activity during inhalation and facilitating activity during exhalation. Further, in a phenomenon known as respiratory sinus arrhythmia (RSA), heat rate increases during inhalation and decreases during exhalation. It is believed that breathing cycles affect heart rate via the ANS with the SNS causing brief acceleration of heart rate and PNS causing deceleration within beat-to-beat intervals through Vagus nerve activity. This is reflected in common deep breathing techniques to promote relaxation and lower heart rate. Studies have shown that exhalation, particularly long duration exhalation, increases HF HRV.

As noted heretofore, an example of the natural variations in the PSD band during normal respiration are illustrated in FIG. 19a. The PSD of a meditator or someone breathing six cycles per minute is shown in FIG. 19a, reflecting that all the available power resides at 0.1 Hz. Many experienced meditators show this oscillation once meditation begins although there is considerable variation in the frequency of the LFO contained within the LF for an individual. Experienced meditators can establish a resonance at 0.02 Hz which is almost VLF, very low frequency. Usually there is an oscillation in the blood pressure observable at this same frequency. Some have called this condition “coherence” and it is generally associated with a calming state because the PSD collapse has only been observed in the LF. Mechanical stimulations on the skin, visual stimulations, auditory stimulations, electrical stimulations, and others will produce the coherent state as long as the stimulations occur between 0.06 and 0.2 Hz; however, the most efficient state with the best peak to peak amplitudes in the systolic blood pressure may be about 0.1 Hz.

The graphs in FIGS. 15 and 16 show the oscillations in blood pressure and heart rate due to the slow-paced abdominal breathing at six Hertz as noted heretofore. The PSDs of the variance show oscillations only at a specific frequency with all of the spectral power having been collapsed into one LF resonant state as illustrated in FIG. 16.

Low frequency paced breathing can also cause the power in the HF band to diminish and the power in the LF band to increase. With breathing practice and with external stimulators, the low and high frequency oscillations can be made to collapse into a single frequency band usually around 0.1 Hz.

What is claimed is:

1. A device for diagnosing and correcting autonomic nervous system imbalance in vertebrates comprising a wearable detector having a body substantially comprised of at least one flexible substrate and containing:
a. a bladder;
b. a pressure sensor;
c. a detector microprocessor with communication means;
d. a power source member;
e. an exterior side;
f. a user contact side; and
g. at least one securing member
wherein said at least one securing member is configured to envelop at least one of the following: a thumb, a finger, a wrist, an ankle, and scalp.
wherein the bladder is placed adjacent an artery and secured by the at least one securing member measure pulsatile waveform and detect inter-beat intervals (IBI) to be read by the pressure sensor;
wherein the detector microprocessor uses IBI data to compute power spectral density (PSD) data comprising an evolving autonomic nervous system spectra for evaluating parasympathetic tone; and
wherein said PSD data is transmitted to at least one communicatively coupled external device.

2. The device of claim 1 wherein said at least one external device is selected from the group of a user device, a stimulation unit, and a respiration monitor.

3. The device of claim 2 wherein said at least two of said at least one external device are communicatively coupled to each other.

4. The device of claim 2 wherein said stimulation unit comprises a mechanical vibrational element placed at a vagus nerve and a heart rate variability monitor having a stimulation unit microprocessor and communication means to receive data.

5. The device of claim 2 wherein the stimulation unit microprocessor controls application of stimulation based on received data, said data including instructions for when to engage stimulation, duration of stimulation, intensity of stimulation, and frequency of stimulation.

6. The device of claim 5 wherein said instructions are transmitted from said user device to said stimulation unit.

7. The device of claim 1 wherein the communication means is at least one from the group of wired, Bluetooth, WiFi, NFC, RFID, or other wireless communication.

8. The device of claim 1 wherein one of the at least one flexible substrate is translucent, and further comprises a thumb pump, LED and closure members and wherein the one of the at least one flexible substrate comprises:
a. on the user contact side:
a. the bladder, the bladder being welded onto the one of at least one substrate,
b. the pressure sensor, the pressure sensor being connected to the bladder to read pulsatile waveform within the bladder and transmit the IBI data to the microprocessor,
c. the LED, the LED being connected to the pressure sensor and indicating when the bladder reaches usable pressure to detect the inter-beat intervals, and
d. the detector microprocessor, the detector microprocessor receiving the IBI data from the pressure sensor, computing PSD data from said IBI data, and communicating the PSD data to predetermined remote microprocessor of said at least one external device, and
e. a power source to power the detector microprocessor and LED, and
b. on the exterior side:
a. the thumb pump positioned over the bladder and used to expand the bladder to the usable pressure,
wherein the securing members interact with one another to affix the device adjacent the artery.

9. The device of claim 1 wherein one of the at least one flexible substrate is translucent, and further comprises an LED and external securing means and wherein the one of the at least one flexible substrate comprises:
a. on the user contact side:
a. the bladder, the bladder being welded onto the one of at least one substrate,
b. the pressure sensor, the pressure sensor being printed directly onto the bladder to pulsatile waveform within the bladder,
c. the LED, the LED being connected to the pressure sensor and indicating when the bladder reaches usable pressure to detect the inter-beat intervals,
d. the detector microprocessor, the detector microprocessor receiving the IBI data from the pressure sensor, computing PSD data from said IBI data, and communicating the IBI data to a predetermined remote microprocessor of said at least one external device.
e. the power source, the power source being connected to the pressure sensor, microprocessor and LED; and
wherein the one of at least one substrate has a tab at a first end and a slot at a second dimensioned to receive the tab for tightening and the external securing means are secured around the wearable detector.

10. The device of claim 1 wherein the at least one flexible substrate is translucent, and further comprises an LED, an on off switch and charging members and wherein:
a. a first of the at least one substrate contains the bladder welded onto a first surface;
b. a second of the at least one substrate contains the charging members on a first surface,
c. a third of the at least one substrate contains the pressure sensor, the microprocessor, LED, power source and a second communication member on a first surface,
wherein the third of the at least one substrate is extended beyond the first and second of the at least one substrate to provide securing a securing member; and
wherein the first side of the third of the at least one substrate is placed adjacent a second side of the second of the at least one substrate and the first side of the second of the at least one substrate is placed adjacent a second side of the first of the at least one substrate to form a finger cuff and then sealed for waterproofing and;
wherein the LED is connected to the pressure sensor to indicate when the bladder reaches usage pressure to detect the inter-beat intervals, and data from the pressure sensor is sent to the microprocessor and then to a predetermined remote microprocessor.

11. The device of claim 1 wherein the at least one flexible substrate is translucent, and further comprises an LED, and an on/off switch and wherein:
a. a first of the at least one substrate has a first periphery and contains the bladder welded onto a first surface and an air channel extending through;
b. a second of the at least one substrate has a second periphery less than the first periphery and contains the pressure sensor positioned adjacent the air channel, the microprocessor, LED, power source and on/off switch;
c. a third of the at least one substrate has a U-shaped configuration with a flat rim having a first periphery and an interior periphery dimensioned to receive the second of the at least one substrate
wherein the flat rim of the third of the at least one substrate is placed adjacent a second side of the first of the at least one substrate and then sealed for waterproofing and;
wherein the LED is connected to the pressure sensor to indicate when the bladder reaches usable pressure to detect the inter-beat intervals, and data from the pressure sensor is sent to the microprocessor for analysis and then to a predetermined remote microprocessor.

12. The device of claim 1 wherein the at least one flexible substrate is translucent, and further comprises an LED, and an on/off switch and wherein:
a. a first of the at least one substrate has a first periphery and contains the bladder welded onto a first surface and an air channel extend therethrough;
b. a second of the at least one substrate contains the pressure sensor positioned adjacent the air channel, the microprocessor, LED, power source and on/off switch;
wherein the first side of the second of the at least one substrate is placed adjacent a second side of the first of the at least one substrate and then sealed for waterproofing and;
wherein the LED is connected to the pressure sensor to indicate when the bladder reaches usable pressure to detect the inter-beat intervals, and data from the pressure sensor is sent to the microprocessor for analysis and then to a predetermined remote microprocessor.

13. A system for diagnosing and correcting autonomic nervous system imbalance, the system comprising:
a wearable detector configured to continuously detect IBIs from a user and compute PSD data from said IBIs using at least one pulse pressure sensor and at least one microprocessor, said IBIs obtained through an observation period in the range of about 3 to 5 minutes at a sampling frequency of at least 300 Hz, said PSD data comprising an evolving autonomic nervous system (ANS) spectra;
a vibratory stimulator configured to deliver vibrations to vagus nerve endings;
a user device communicatively coupled to at least the wearable device and configured to run applications, display information on a graphical user interface, store information in at least one database locally and remotely, and transmit information;
one or more processors communicatively coupled with at least the wearable detector and the user device, the one or more processors configured to:
transmit PSD data to display on said user device providing updated ANS status information to the user;
analyze PSD data for indicators of ANS imbalance correlating to one or more health-related conditions, said indicators comprising one or more of the following: change in low frequency power (LF) to high frequency power (HF) ratio, frequency power readings within a pre-determined pattern, and erratic frequency power readings;
compare indicators to information stored in said at least one database;
determine recommended therapeutic action based on information stored in said at least one database, said recommended therapeutic action comprising one or more of the following: paced breathing, vibrational stimulation of the vagus nerve, and neuromodulator application;
transmit instructions for recommended action to at least display on the user device;
receive additional PSD data for continued analysis during application of therapeutic action; and
record ANS changes based on said additional PSD data and save ANS changes to said at least one database.

14. The system of claim 13, wherein said recommended therapeutic action comprises paced breathing at 10 bpm plus vibrational stimulation of the vagus nerve during exhalation to strengthen HF.

15. The system of claim 13, wherein said one or more processors are further communicatively coupled to said vibratory stimulator and configured to transmit instructions for recommended action to said vibratory stimulator to automatically prompt vibrational stimulation of the vagus nerve.

16. The system of claim 13, further comprising a respiratory monitor transmitting respiration data to said at least one processor, said respiration data used to determine type and timing of recommended therapeutic action.

17. The system of claim 13, wherein the one or more health-related conditions comprise vasomotor symptoms, anxiety, and PTSD.

18. A method of correcting ANS imbalance comprising:
i) Computing PSD data based on IBIs detected by a wearable detector;
ii) Transmitting said PSD data to an external device;
iii) Analyzing PSD data to identify ANS imbalance indicators based on PSD data;
iv) Comparing ANS imbalance indicators to like information in database;
v) Determining recommended corrective therapy using one or more techniques chosen from paced breathing, vibrational stimulation, neuromodulation;
vi) Providing recommended therapy to user via electronic display;
vii) Applying recommended therapy;
viii)Continuously updating PSD data during therapy;
ix) Recording results of therapy application to store on database.
